(19) United States
(12) Patent Application Publication
(10) Pub. No.: US 2006/0240126 A1
(43) Pub. Date: Oct. 26, 2006

(54) COMPOSITIONS CONTAINING ALLIUM SATIVUM LINN. (GARLIC) NATURALLY ENRICHED WITH ORGANIC SELENIUM COMPOUNDS FOR NUTRITIONAL SUPPLEMENTATION

(76) Inventors: Muhammed Majeed, Piscataway, NJ (US); Rajinder Kumar Bammi, Bangalore (IN); Vladimir Badmaev, Piscataway, NJ (US); Subbalakshmi Prakash, Piscataway, NJ (US); Kalyanam Nagabhushanam, Piscataway, NJ (US)

Correspondence Address: SABINSA CORPORATION, 70 ETHEL ROAD WEST, UNIT 6, PISCATAWAY, NJ, 08854, US

(21) Appl. No.: 11/164,787

(22) Filed: Dec. 6, 2005

Related U.S. Application Data
(63) Continuation-in-part of application No. 10/605,578, filed on Oct. 9, 2003, now Pat. No. 7,014,874.

(51) Int. Cl. A61K 36/8962; A61K 33/04

(52) U.S. Cl. 424/702; 424/754

(57) ABSTRACT

The invention discloses selenium enriched garlic compositions that are a safe and efficacious means of providing supplemental amounts of the essential trace mineral nutrient selenium, to humans and animals.

### CROSS REFERENCE TO RELATED APPLICATIONS

This application claims the benefit of U.S. Provisional patent application No. 60/367,274 filed on Mar. 26, 2002, and U.S. patent application Ser. No. 10/249,239, filed on Mar. 25, 2003, and is a continuation-in-part of U.S. patent application Ser. No. 10/605,578, filed on Oct. 9, 2003 the disclosure of which is hereby incorporated by reference.

### BACKGROUND OF INVENTION

The present invention is related to compositions of garlic naturally enriched with organic selenium compounds, and the use of these compositions for nutritional supplementation in humans and animals.

Selenium is a vital trace element nutrient with multiple roles in the growth and functioning of living cells in higher animals and humans. At the molecular level, selenium (as selenocysteine) is an essential component of the active sites of the antioxidant enzyme glutathione peroxidase, and the enzymes participating in thyroid functions iodothyronine-5′-deiodinase and mammalian thioredoxin reductase. Selenium is also present in several other mammalian selenoproteins. Low selenium status has been linked with the occurrence of decreased immunity to diseases and the prevalence of various forms of cancer.

Selenium occurs in foods, through uptake by plants from the soil, in the form of the selenoamino acids, selenomethionine and selenocysteine, and their derivatives. However, as the element is unevenly distributed in the earth's crust, dietary supplementation is often needed. Organic selenium compounds are preferred for supplementation on account of their superior bioavailability. An ideal nutritional supplement would be a selenium enriched edible plant part wherein the selenium metabolically accumulates in the form of bioavailable organic selenium compounds. Plants that naturally contain higher levels of the sulfur containing amino acids such as those from the Allium and Brassica species are preferred for enrichment, based on metabolic criteria. Selenium-enriched garlic is reported to be useful as a nutritional supplement in the prevention of cancer (Ip, et al., 1992, 1996; Ip and Lisk, 1993, 1997; Lu et al., 1996).

Clinical intervention trials and in vitro data revealed the efficacy of selenium in the form of selenium yeast or selenomethionine in cancer prevention (Clark et al., 1996, 1998). Ip et al (2000) described the role of chemical speciation on the comparative activity of selenium-enriched garlic and selenium yeast in mammary cancer prevention in rats, wherein selenium-enriched garlic was shown to be more efficacious than selenium yeast. γ-glutamyl Se-methylselenocysteine is reported to be the major form of selenium in selenium-enriched garlic while L-Selenomethionine was shown to be the major form of selenium in selenium enriched yeast. However, the present inventors have for the first time identified Allylselenocysteine as yet another major selenoamino acid present in selenium enriched garlic bulbs.

Laboratory studies indicate that γ-Glutamyl-Se-methyl-L-selenosysteine is an effective chemopreventive agent, serving as a carrier for Se-Methyl-L-selenocysteine (Dong et al., 2001; Medina et al., 2001). Se-Methyl-L-selenocysteine is a well researched chemopreventive organoselenium compound, which is not incorporated in the body proteins, and is therefore less toxic than other forms of supplemental selenium (Ip et al., 1994; Medina et al., 2001).

U.S. patent application Ser. No. 10/249,239 disclosed a novel process for preparing garlic bulbs naturally enriched with an unique composition of organic selenium compounds through a soilless culture technique. U.S. patent application Ser. No. 10/605,578 described a process for preparing a concentrate containing organic selenium compounds extracted from enriched garlic bulbs of U.S. patent application Ser. No. 10/249,239, for nutritional supplementation in humans and animals.

### SUMMARY OF INVENTION

The current invention discloses compositions of naturally enriched garlic for nutritional supplementation in humans and animals, comprising organic selenium compounds from the methods disclosed in U.S. patent application Ser. Nos. 10/249,239 and 10/605,578.

### DETAILED DESCRIPTION

The invention discloses compositions comprising Methylselenic acid, Allyl Selenocysteine, the Selenoamino acids L-Selenomethionine and Se-Methyl-L-selenocysteine; and Selenoamino acid dipeptides, N-γ (L-Glutamyl) L-selenomethionine, and N-γ (L-Glutamyl) Se-methyl-L-selenocysteine, A dispersion of these compounds is made in natural garlic powder containing 10 to 500 ppm of Alliin, for nutritional supplementation.

### Example 1

### Process to Prepare a Composition Containing Selectively Fractionated Bioactive Organic Selenium Compounds from Selenium-Enriched Allium sativum Linn Bulbs:

100 kg of selenium-enriched garlic bulbs prepared by soilless culture technique were crushed and subjected to multi-stage supercritical fluid extraction followed by chromatographic separation. High pressure carbon dioxide (10 to 60 MPa), modified with ethanol and water (50:50), was used to extract Selenium containing non-protein amino acids as well as selenoamino acid dipeptides. These were separated and purified using preparative HPLC, the mobile phase and water were removed by evaporation under reduced pressure and freeze drying, to yield bioactive selenoamino acid and selenoamino acid dipeptide fractions. The fractions obtained were blended with natural garlic powder to yield a composition containing 100 to 2000 ppm of selenium in the form of organic selenium compounds.

An exemplary composition of the selenium enrichment concentrate configured to provide 1000 ppm organic selenium content in natural garlic powder containing 200 ppm Alliin is detailed in Table 1.

TABLE 1
Composition of the Enrichment Concentrate

| | | Elemental |
|---|---|---|
| | | selenium |
| | Level in | equivalent |
| Component | ppm | (ppm) |
| Methylselenic Acid | 25 | 15.6 |
| Allylselenocysteine | 378 | 143.6 |
| Se-methyl-L-selenocysteine | 120 | 52.3 |
| L-selenomethionine | 75 | 30.9 |
| N-Υ (L-Glutamyl) L-selenomethionine | 60 | 15.0 |
| N-Υ (L-Glutamyl) Se-methyl-L- | 344 | 87.0 |
| selenocysteine | 000 | 000.0 |
| Total = | 1002 | 344.4 |

### Example 2

### Toxicological Studies on the Selenium Enriched Garlic Powder

The study was designed to determine the acute oral toxicity of garlic powder enriched with the mixture of Example 1, to Sprague Dawley rats (male and female) of age 6 to 8 weeks with weight ranging from 124.1 g to 140.6 g. The rats were randomly selected in groups of five of like sex at the time of initiating the study.

The test substance suspended in water was administered by oral route to rats, at a dose volume of 10 ml/kg. Ten rats (5 male and 5 female) were allocated to a treatment of 2000 mg/kg of the test composition. The rats were observed for 14 days after treatment. All animals survived through the study period of 14 days and were free of intoxicating signs 1 to 2 hours after the treatment. The LD50 value of the composition in rats by oral route was found to be greater than 2000 mg/kg body weight.

### Example 3

### Clinical Efficacy and Safety of the Composition

Study Objective: To Determine the Efficacy and Safety of the Composition in Reducing Oxidative Stress Levels.

Study Design: 42-day open prospective single centre study to measure the efficacy and safety of composition. In this 42 day study, each patient was administered the composition (providing 100 mcg of elemental selenium) in a single dose, daily after a meal. Selection of Subjects: The selection criteria stipulated were that the subjects have a BMI (body mass index) of 20-38 kg/m2, fall in the age group of 35 to 60 years, with an increased risk of heart disease. These subjects would be otherwise in good health, with no history of alcohol or drug abuse, and no known allergies. Kidney and liver functions would be normal. 13 subjects (in the age group of 25 to 43 years (6 males and 7 females)), participated in the study.

Study Procedures: Efficacy was assessed based on change in oxidative stress, as evidenced by measuring antioxidant profile. Safety was assessed based on standard liver function and kidney function tests. Adverse events, if any, were recorded. Liver function is assessed by using specific enzyme markers. For example, both SGPT (Serum Glutamate-Pyruvate Transaminase) and SGOT (Serum Glutamate-Oxaloacetate Transaminase) are important in the clinical diagnosis of disease. These enzymes, particularly abundant in heart and in liver, are released from injured cells in myocardial infarction, infections, liver damage or cardiovascular disease. Kidney function tests seek to evaluate how well the kidneys filter and transport metabolic waste from the blood into the urine. Serum creatinine, serum urea, and electrolyte (sodium, potassium and chloride) levels are measured. ESR (Erythrocyte sedimentation Rate) and haemoglobin levels, if abnormal, provide an indication of the presence of disease.

Results: Efficacy: From day 0 (baseline) to day 21, and from day 0 to day 42, plasma lipid peroxidation reduced by an average of 5.39 units and 9.28 units respectively. Both decreases were statistically significant. Lymphocyte antioxidant count for Glutathione-S-transferase and Superoxide dismutase were found to increase from day 0 to day 21, and from day 0 to day 42. The Catalase values decreased during this period. All the mean differences from baseline were found to be statistically significant.

Safety: The laboratory values of parameters for liver function and kidney function were found to be within admissible limits, with minor changes reported during the course of the study. The variation was within 5% of normal values. Similarly blood lipid profiles remained within the normal range. No adverse events were observed in any of the subjects or reported during the study.

Conclusions: The results of this open prospective clinical study suggest that the composition effectively reduces oxidative stress levels, and is safe for use as an antioxidant nutritional supplement.

While an exemplary embodiment of the composition and its use is presented in the current invention, the intention is to cover all modifications and alterations falling within the scope of the appended claims.

What is claimed is:

1. Garlic enriched with a concentrate of selenium compounds for nutritional supplementation comprising:
from about 10 ppm to about 500 ppm of methylselenic acid;
from about 10 ppm to about 500 ppm of allyl selenocysteine;
from about 10 ppm to about 500 ppm of L-selenomethionine;
from about 10 ppm to about 500 ppm of Se-methyl-L-selenocysteine;
from about 10 ppm. to about 500 ppm of N-γ (L-Glutamyl) L-selenomethionine; and
from about 10 ppm to about 500 ppm of N-γ (L-Glutamyl) Se-methyl-L-selenocysteine.

2. The selenium enriched garlic supplement of claim 1 wherein the supplement is further comprised of from about 10 ppm to about 500 ppm alliin.

3. The selenium enriched garlic supplement of claim 1 wherein the supplement is in powder form and provides about 100 ppm to about 2000 ppm of elemental selenium.

4. The selenium enriched garlic supplement of claim 1 wherein the supplement is further comprised of a pharmaceutically acceptable carrier material.

5. The selenium enriched garlic supplement of claim 1 which is administered orally once per day to a human or animal subject for general health maintenance.

6. The selenium enriched garlic supplement of claim 1 wherein the total daily dosage is divided and administered in portions during the day to humans and animals.

7. The selenium enriched garlic supplement of claim 1 wherein the dosage form is compressed into a tablet or filled into hard or soft gelatin capsule, or added to a food product.
